(19) United States
(12) Plant Patent Application Publication
(10) Pub. No.: US 2008/0155725 P1
(43) Pub. Date: Jun. 26, 2008

(54) LOBELIA PLANT NAMED 'LOBTRAMIDBLU'

(75) Inventors: Johannes Simon Nicolaas Oud, Bovenkarspel (NL); Theodorus Cornelis Maria van Kleinwee, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/644,923

(22) Filed: Dec. 22, 2006

(51) Int. Cl. A01H 5/00

(52) U.S. Cl. PLT/451

(57) ABSTRACT

A new Lobelia plant particularly distinguished by its spreading plant habit, strong branching, large flower size, and blue flower color.

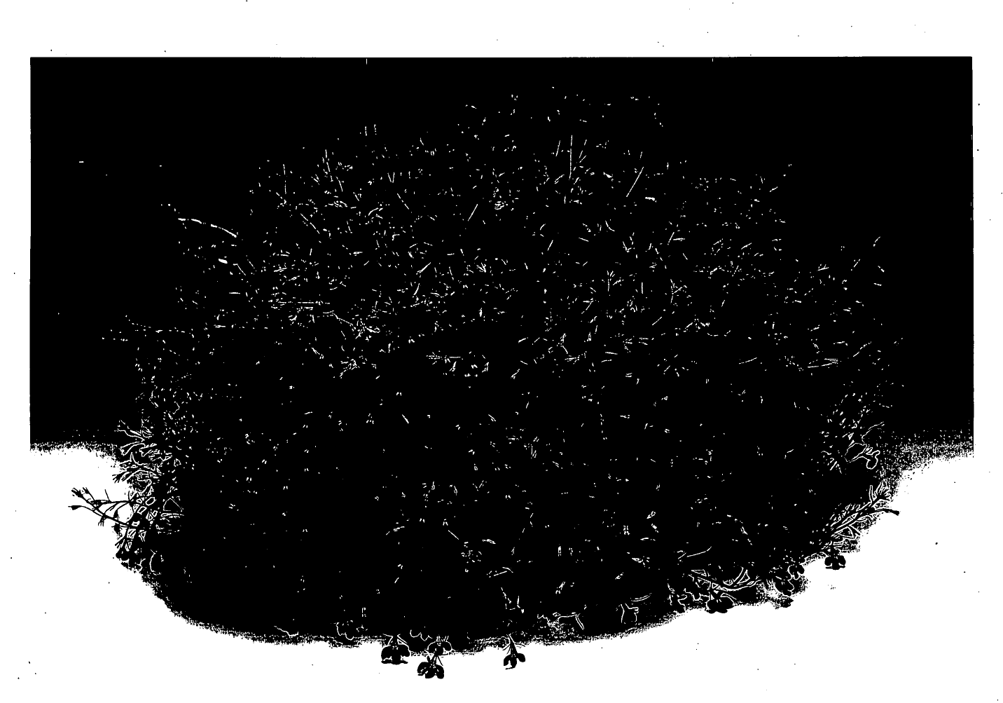

### LATIN NAME OF THE GENUS AND SPECIES OF THE PLANT CLAIMED

Lobelia erinus

### VARIETAL DENOMINATION

‘Lobtramidblu’

### BACKGROUND OF THE NEW PLANT

The present invention relates to a new and distinct cultivar of Lobelia plant. The new cultivar is propagated by cuttings resulting from the cross between Lobelia erinus ‘A0924-1’ and Lobelia erinus ‘A0951-1.’ ‘A0951-1’ is a blue flower color selection of our own breeding, having a spreading habit, very strong growth, good branching plant habit and being early flowering. ‘A0924-1’ is a blue flower color selection of our own breeding, having a medium growth, trailing plant habit and being rather early flowering. Neither ‘A0951-1’ nor ‘A0924-1’ has been patented.

As a result of this cross the present cultivar was created in 2001 in Enkhuizen, Netherlands and has been repeatedly asexually reproduced by cuttings in Enkhuizen over a five year period. It has retained its distinctive characteristics through successive propagations and is firmly fixed.

### DESCRIPTION OF THE PHOTOGRAPH

This new Lobelia plant is illustrated by the accompanying photograph which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Lobelia. The data, which defines these characteristics, were collected from asexual reproductions carried out in Enkhuizen, Netherlands. The plant history was taken on 30 week old plants.

TABLE 1
DIFFERENCES BETWEEN ‘LOBTRAMIDBLU’
AND TWO SIMILAR CULTIVARS

| | | | ‘Wesstar’ | ‘Ricardii’ |
|---|---|---|---|---|
| | | | (U.S. Plant Patent | (Not |
| Characteristic | | ‘Lobtramidblu’ | No. 12,678) | patented) |
| Plant | Height | 15 cm | 20 cm | 30 cm |
| | Width | 45 cm | 50 cm | 60 cm |
| | Shape | Upright to | Spreading | Spreading |
| | | trailing | | |
| Flower | Upper surface | 94A | 96A with spots | 94A |
| color | | | darker than 95A | |
| | Lower surface | 94B | 96C | 94B |
| | Flower tube | 94A | 94A | 94B |

No descriptions of parentlines are available. The only info is mentioned as background of the new plant on page 1.
The plant:
   Classification — botanical.—Lobelia erinus.
   Parentage.—Female parent: A seedling named ‘A0924-1’ is one of our seedlings from our A-generation of plants bred in 1999. Male parent: A seedling named ‘A0951-1’ is one of our seedlings from our A-generation of plants bred in 1999.
   Growth habit.—Upright to trailing.
   Plant height.—15 cm.
   Spreading area of plant.—45 cm.
   Growth rate.—Medium.
   Strength.—Good.
   Branching character.—Freely branching and lateral branching at every node Pinching will enhance branching. Branches strong and flexible.
   Blooming period.—From May until October.
The stem:
   Diameter.—2.5 mm.
   Length.—25 cm.
   Texture.—Old stem: glossy, Young stem: smooth.
   Color.—RHS 137C in old stems, RHS 144B in young stems.
   Length of internode.—0.5-4 cm, depending on the light conditions where the plant is grown, and the age of the stem.
   Shape.—(Tri)angular.
The foliage:
   Shape.—Leaves simple, generally symmetrical and flat. Broadly ovate with rounded apex, attenuate base and dentate margin in leaves of juvenile plant; more lanceolate in leaves of mature plant with entire margin.
   Texture.—Smooth on upper and lower side.
   Length.—2.5 cm (leaves of juvenile plant up to 4 cm, leaves of mature plant up to 3 cm).
   Width.—In leaves of juvenile plant 2 cm, leaves of mature plant up to 1 cm.
   Color.—Upper surface: RHS 146A in old leaves, near RHS 143A in young leaves Lower surface: RHS 147C in old leaves, RHS 146B in young leaves Foliage color applies for juvenile as well as mature phase.
   Veins.—pinnate, upper side RHS 143C, lower side RHS 143C.
   Length of petiole.—Not present.
The flower:
   Type and habit.—Flower form at apical axils, usually one flower per axil. Flowers labiate, upper lip consisting of two small petals and lower lip consisting of three larger and broader petals. Petals have obovate shape with a mucronulate apex and entire margin.
   Lastingness on the plant.—10-14 days depending on temperature. Petals are fused at base into a tube. Freely and continuously flowering, around 100-150 flowers. Flowers face outward.
   Width — petal.—Upper petals 0.1 cm, lower petals 0.5 cm.
   Length — petal.—Upper petals 0.6 cm, lower petals 0.8 cm.
   Depth — flower.—1.7 cm.
   Diameter — flower.—1.5 cm.
   Texture.—Very lightly pubescent, upper and lower side.
   Buds.—Length: 1.2 cm Diameter: 0.8 cm Form: Tubular Color: Intensity varies from RHS 90A to 90D.
   Flower color.—Upper surface: RHS 94A Lower surface: RHS 94B Flower tube (outside): RHS 94A with vein N88B on upper side, 94C on lower side with spots 94B Remarks: On the three lower petals of flower, there is a white spot, width 0.3 cm, depth 0.3 cm, color whiter than RHS 155D. There is not a white spot on the upper petals.
   Throat.—Texture: Whiskered Depth: 0.6 cm Diameter: 0.3 cm Color: whiter than RHS 155D with spots darker than RHS 94A.
   Sepals.—No. of sepals: 5 Length: 0.8 cm Width: 0.5 mm Texture: Smooth Color: RHS 200B upper side and RHS 200B lower side Shape: Linear Apex: Acute Margin: Entire Sepals are fused at base.
   Flower stem.—Length: 1.2-2 cm Angle: 45° Strength: Rigid, flexible Color: RHS 137B occasionally some anthocyan 200B Diameter: 0.5 mm.
Reproductive organs:
   Androecium.—Stamen number: 5 Anther shape: Oblong Anther length: 2.5 mm Anther color: RHS 200A Pollen color: RHS 8B Amount of pollen: Moderate.
   Gynoecium.—Pistel length: 0.8 cm Stigma shape: Bilobate Stigma diameter: 0.8 mm Stigma color: RHS N92A Style color: RHS 92A near stigma, RHS 148B towards base. Ovary color: RHS 92A with anthocyaan 200B.
Seed development: Some seed development has been observed on plants of the new Lobelia.
   Seed.—Color: RHS N199B Shape: Round Size: Very small, ‘like dust’ Amount per seed pod: Approximately 2000.
Fruit:
   Size.—Diameter: 0.2 cm Length: 0.6 cm.
   Color.—RHS 200B.
Disease resistance: No susceptibility or resistance to fungal, bacterial or viral pathogens has been noted.

What is claimed is:

1. A new and distinct variety of Lobelia plant, named ‘Lobtramidblu,’ as illustrated and described herein.
